(19) United States
(12) Patent Application Publication
(10) Pub. No.: US 2022/0274913 A1
(43) Pub. Date: Sep. 1, 2022

(54) PROCESS FOR MANUFACTURING A SUBSTITUTED CYCLOHEXANECARBONITRILE

(71) Applicant: SOLVAY SA, Brussels (BE)

(72) Inventors: Karol LORENT, Strépy-Bracquegnies (BE); Juan-Teva CARLIER, Sombreffe (BE); Simon GUELEN, Chabanière (FR)

(21) Appl. No.: 17/637,468

(22) PCT Filed: Sep. 11, 2020
(86) PCT No.: PCT/EP2020/075483

§ 371 Date: Feb. 23, 2022

(30) Foreign Application Priority Data
Sep. 11, 2019 (EP) 19196598.7

(51) Int. Cl. C07C 255/46; C07C 253/00

(52) U.S. Cl. CPC C07C 255/46; C07C 253/00; C07C 2601/14

(57) ABSTRACT

A process for manufacturing a substituted cyclohexanecarbonitrile said process comprising the following steps: —reacting the corresponding substituted cyclohexanecarboxylic acid with thionyl chloride to make the corresponding acyl chloride; and simultaneously or subsequently —reacting the chloride with sulfonamide in sulfolane as solvent to make the substituted cyclohexanecarbonitrile.

The present invention relates to a process for manufacturing a substituted cyclohexanecarbonitrile, to specific substituted cyclohexanecarbonitriles and to their use as solvent in the manufacture of an aqueous hydrogen peroxide solution.

Hydrogen peroxide is one of the most important inorganic chemicals to be produced worldwide. Its industrial applications include textile, pulp and paper bleaching, organic synthesis (propylene oxide), the manufacture of inorganic chemicals and detergents, environmental and other applications.

Synthesis of hydrogen peroxide is predominantly achieved by using the Riedl-Pfleiderer process (originally disclosed in U.S. Pat. Nos. 2,158,525 and 2,215,883), also called anthraquinone loop process or AO (auto-oxidation) process.

This well-known cyclic process makes use typically of the auto-oxidation of at least one alkylanthrahydroquinone and/or of at least one tetrahydroalkylanthrahydroquinone, most often 2-alkylanthraquinone, to the corresponding alkylanthraquinone and/or tetrahydroalkylanthraquinone, which results in the production of hydrogen peroxide.

The first step of the AO process is the reduction in an organic solvent (generally a mixture of solvents) of the chosen quinone (alkylanthraquinone or tetrahydroalkylanthraquinone) into the corresponding hydroquinone (alkylanthrahydroquinone or tetrahydroalkylanthrahydroquinone) using hydrogen gas and a catalyst. The mixture of organic solvents, hydroquinone and quinone species (working solution, WS) is then separated from the catalyst and the hydroquinone is oxidized using oxygen, air or oxygen-enriched air thus regenerating the quinone with simultaneous formation of hydrogen peroxide. The organic solvent of choice is typically a mixture of two types of solvents, one being a good solvent of the quinone derivative (generally a non-polar solvent for instance a mixture of aromatic compounds) and the other being a good solvent of the hydroquinone derivative (generally a polar solvent for instance a long chain alcohol or an ester). Hydrogen peroxide is then typically extracted with water and recovered in the form of a crude aqueous hydrogen peroxide solution, and the quinone is returned to the hydrogenator to complete the loop.

The use of di-isobutyl-carbinol (DIBC) as polar solvent is namely described in Patent applications EP 529723, EP 965562 and EP 3052439 in the name of the Applicant. The use of a commercial mixture of aromatics sold under the brand Solvesso®-150 (CAS no. 64742-94-5) as non-polar solvent is also described in said patent applications. This mixture of aromatics is also known as Caromax, Shellsol, A150, Hydrosol, Indusol, Solvantar, Solvarex and others, depending on the supplier. It can advantageously be used in combination with sextate (methyl cyclohexyl acetate) as polar solvent (see namely U.S. Pat. No. 3,617,219).

Most of the AO processes use either 2-amylanthraquinone (AQ), 2-butylanthraquinone (BQ) or 2-ethyl anthraquinone (EQ). Especially in the case of EQ, the productivity of the working solution is limited by the lack of solubility of the reduced form of ETQ (ETQH). It is namely so that EQ is largely and relatively quickly transformed in ETQ (the corresponding tetrahydroalkylanthraquinone) in the process. Practically, that ETQ is hydrogenated in ETQH to provide H₂O₂ after oxidation. The quantity of EQH produced is marginal in regards of ETQH. It means that the productivity of the process is directly proportional to the amount of ETQH produced. The reasoning is the same for a process working with AQ or BQ instead of EQ.

The hydrogenated quinone solubility issue is known from prior art and some attempts were made to solve it. Namely co-pending PCT application EP2019/056761 to the Applicant, discloses the use of non-aromatic cyclic nitrile type solvents as polar solvent in the mixture, more specifically the use of cyclohexanecarbonitriles, and especially substituted ones (in which the nitrile function is protected from chemical degradation).

The synthesis of such solvents has been reported in literature. For instance 2,2,6-trimethyl-cyclohexane-carbonitrile was synthesized by Shive et al. (JACS, 1942, vol. 64, pp. 385-389) starting from geranic acid which was first cyclized using formic acid (1); was then hydrogenated to the corresponding saturated acid (2,2,6-trimethylcyclohexanecarboxylic acid) (2), which was then transformed in the corresponding acyl chloride using thionyl chloride (3), then in the corresponding amide using ammonia (4) and finally, in the corresponding carbonitrile by dehydration using phosphorus pentoxide (5).

This way of synthesis hence implies 5 reaction steps. The idea behind the present invention is to reduce this number of steps namely by directly synthesizing the cyclohexanecarbonitrile starting from the corresponding carboxylic acid using thionyl chloride and sulfonamide in sulfolane.

Therefore, in a first aspect, the present invention relates to a process for manufacturing a substituted cyclohexanecarbonitrile said process comprising the following steps:
reacting the corresponding substituted cyclohexanecarboxylic acid with thionyl chloride to make the corresponding acyl chloride; and simultaneously or subsequently
reacting the chloride with sulfonamide in sulfolane as solvent to make the substituted cyclohexanecarbonitrile.

The substituent(s) on the carbon skeleton of cyclohexanecarbonitrile according to the invention is/are preferably alkyl group(s), preferably methyl and/or ethyl group(s). Preferably, there are at least 2 methyl groups as substituents in order to protect the nitrile, more preferably at least 3 and most preferably at least 4 methyl groups. Another alternative would be to use at least one propyle, diethyle, methylisopropyle, butyle, t-butyle . . . the latter being preferred. The substituent group(s) attached to the hydrocarbon cycle preferably is close to the nitrile function in order to protect it, typically in position 1, 2 and/or 6.

The substituted cyclohexanecarboxylic acid is preferably obtained by hydrogenating one of the corresponding substituted cyclohexenecarboxylic acids with hydrogen gas in the presence of a hydrogenation catalyst for instance based on Ni, Pd or Pt. Each of these metal catalysts is preferably prepared in a special way and/or in a given form:
nickel is usually used in a finely divided form called “Raney nickel” and which is prepared by reacting a Ni—Al alloy with NaOH;
palladium is generally obtained commercially “supported” on an inert substance, such as charcoal (i.e. as a Pd/C catalyst) and ethanol is generally chosen as solvent in this case;
platinum is generally used as PtO2, also called Adams' catalyst, although it is actually platinum metal that is the catalyst; the hydrogen used to add to the carbon-carbon double bond also reduces the platinum(IV) oxide to finely divided platinum metal. Ethanol or acetic acid is generally used as solvent with this catalyst.

Good results are obtained in the frame of the invention with PtO2 (i.e. the Adams' catalyst) as catalyst and acetic acid (preferably glacial acetic acid) as solvent. Hydrogenation preferably takes place at a temperature from ambient to 100° C., preferably from 30 to 80° C., more preferably from 40 to 60° C., a temperature about 50° C. giving good results in practice. Hydrogenation preferably takes place at a pressure from atmospheric to 20 bar, the higher the substitution degree the higher the pressure. The concentration used (in weight) is preferably from 10 to 50% of the organic in the solvent, more preferably of 20 to 30% of the organic in the solvent, good results being obtained with 25% of the organic in the solvent.

In a first embodiment of the present invention, the starting substituted cyclohexenecarboxylic acid is obtained by cyclization of the corresponding linear acid in the presence of a catalyst like phosphoric acid eventually in toluene, or BF3 etherate. When the catalyst is phosphoric acid in toluene, good results are obtained with from 5 to 100% molar phosphoric acid, more preferably from 10 to 50% molar, about 20% molar giving good results in practice. This reaction is preferably conducted at the reflux temperature of toluene (110° C.) at atmospheric pressure.

Good results are obtained for this embodiment when the linear acid is geranic acid, the catalyst is phosphoric acid in toluene and the resulting substituted cyclohexanecarbonitrile is 2,2,6-trimethylcyclohexanecarbonitrile (C10A).

In a second embodiment, the starting substituted cyclohexenecarboxylic acid is obtained by a Diels-Alder reaction between a conjugated diene and an unsaturated carboxylic acid in the presence of a Lewis acid catalyst such as ZnCl2, BF3, BCl3, BoB(Ac)4 (tetraacetyl diborate), SnCl4, AlCl3, TiCl4, TiCl2-isopropoxide and rare earth derivatives like ytterbium trichloride, triflate or triflamide, preferably in a solvent like THF. Although Diels-Alder reactions may occur simply by thermal activation, Lewis acid catalysis enables them to proceed at low temperatures, i.e. without any thermal activation, with a shorter reaction time and with a different regioselectivity.

In a first sub-embodiment, the conjugated diene is 2,4-dimethylpenta-1,3-diene, the unsaturated carboxylic acid is methacrylic acid, the Lewis acid catalyst is BoB(Ac)4, THF is used as solvent and the desired resulting substituted cyclohexanecarbonitrile is 1,2,2,4-tetramethylcyclohexanecarbonitrile (C11B). In practice, a mixture of isomers can be obtained hence comprising besides 1,2,2,4-tetramethylcyclohexanecarbonitrile, the 1,3,3,5 isomer.

In a second sub-embodiment, the conjugated diene is 2,4-dimethylpenta-1,3-diene, the unsaturated carboxylic acid is crotonic acid, the Lewis acid catalyst is BoB(Ac)4, THF is used as solvent and the desired resulting substituted cyclohexanecarbonitrile is 2,2,4,6-tetramethylcyclohexanecarbonitrile (C11C). In practice, a mixture of isomers can be obtained hence comprising besides 2,2,4,6-tetramethylcyclohexanecarbonitrile, the 2,3,3,5 isomer.

In a third sub-embodiment, the conjugated diene is 2,3-dimethylbuta-1,3-diene, the unsaturated carboxylic acid is tiglic acid, the Lewis acid catalyst is BoB(Ac)4, THF is used as solvent and the resulting substituted cyclohexane carbonitrile is one of the stereoisomers of 1,2,4,5-tetramethylcyclohexanecarbonitrile (C11D).

In a fourth sub-embodiment, the conjugated diene is 2,3-dimethylbuta-1,3-diene, the unsaturated carboxylic acid is angelic acid, the Lewis acid catalyst is BoB(Ac)4, THF is used as solvent and the resulting substituted cyclohexane carbonitrile is another stereoisomer of 1,2,4,5-tetramethylcyclohexenecarbonitrile (C11E).

In a fifth sub-embodiment, the conjugated diene is 2,4-dimethylpenta-1,3-diene, the unsaturated carboxylic acid is tiglic acid, the Lewis acid catalyst is BoB(Ac)4, THF is used as solvent and the resulting substituted cyclohexenecarbonitrile is 1,2,3,3,5-pentamethylcyclohexanecarbonitrile (C12A).

Reactions are preferably carried-out between 20° C. and 100° C., more preferably between 40° C. and 60° C., a temperature about 50° C. giving good results in practice. The catalyst used is preferably in a concentration between 1 and 50% molar, more preferably between 5 and 15% molar, about 10% molar giving good results in practice. The dilution by solvent used is preferably between 5 and 50% by weight, more preferably between 10 and 30%, about 20% by weight giving good results in practice.

The substituted cyclohexanecarboxylic acid can also be obtained by hydrolysing the corresponding substituted cyclohexane ester, which can for instance be obtained by hydrogenating the corresponding substituted cyclohexene ester with hydrogen gas in the presence of a hydrogenation catalyst preferably as described above.

In one embodiment, the substituted cyclohexane ester is ethyl 2,2,5,6-tetramethylcyclohexanecarboxylate, the substituted cyclohexene ester is ethyl 2,3,6,6-tetramethylcyclohex-2-enecarboxylate and the resulting substituted cyclohexane carbonitrile is 2,2,5,6-tetramethylcyclohexanecarbonitrile (C11A).

Possible reaction steps to obtain the ethyl 2,3,6,6-tetramethylcyclohex-2-enecarboxylate are the following:
a second order nucleophilic substitution reaction (SN2) is carried out on ethyl 3-oxo-2-methylbutanoate (so called 2-methylacetoacetate) with 1-chloro-3-methylbut-2-ene in alkaline medium. In such conditions, saponification occurs and the subsequent decarboxylation of the β-ketoacid affords the compound 3,6-dimethylhept-5-en-2-one.
a Horner-Wadsworth-Emmons (HWE) reaction is carried out on 3,6-dimethylhept-5-en-2-one with triethylphosphonoacetate and sodium hydride to form the corresponding ester ethyl 3,4,7-trimethylocta-2,6-dienoate
the cyclization of ethyl 3,4,7-trimethylocta-2,6-dienoate with a Lewis acid catalyst or with phosphoric acid in toluene (as described above) to obtain ethyl 2,3,6,6-tetramethylcyclohex-2-enecarboxylate.

The present invention also concerns specific substituted cyclohexanecarbonitriles which can namely be obtained by the above described process namely compounds C11A, C11B, C11C, C11D, C11E and C12A as described above. The synthesis of these compounds has not been reported yet in literature and compared to their C10 homologues (which are known: see the above mentioned co-pending PCT application to the Applicant), they give better results in terms of hydrogenated quinone solubility and are less water extractable hence leading to less TOC in the final hydrogen peroxide solution.

Finally, the present invention also relates to the use of these novel compounds as polar organic solvent in a process for manufacturing an aqueous hydrogen peroxide solution. More specifically, it relates to a process comprising the following steps:
hydrogenating a working solution which comprises an alkylanthraquinone and/or tetrahydroalkylanthraquinone and a mixture of a non-polar organic solvent and a polar organic solvent;
oxidizing the hydrogenated working solution to produce hydrogen peroxide; and
isolating the hydrogen peroxide,

wherein the polar organic solvent is obtained by a process as described above and/or has the formula C11A, C11B, C11C, C11D, C11E or C12A as described above.

The term “alkylanthraquinone” is intended to denote a 9,10-anthraquinone substituted in position 1, 2 or 3 with at least one alkyl side chain of linear or branched aliphatic type comprising at least one carbon atom. Usually, these alkyl chains comprise less than 9 carbon atoms and, preferably, less than 7 carbon atoms. Examples of such alkylanthraquinones are ethylanthraquinones like 2-ethylanthraquinone (EQ), 2-propylanthraquinone, 2-sec- and 2-tert-butylanthraquinone (BQ), 1,3-, 2,3-, 1,4- and 2,7-dimethylanthraquinone, amylanthraquinones (AQ) like 2-iso- and 2-tert-amylanthraquinone and mixtures of these quinones.

The term “tetrahydroalkylanthraquinone” is intended to denote the 9,10-tetrahydroquinones corresponding to the 9,10-alkylanthraquinones specified above. Hence, for EQ and AQ, they respectively are designated by ETQ and ATQ, their reduced forms (tetrahydroalkylanthrahydroquinones) being respectively ETQH and ATQH.

Preferably, an AQ or EQ is used, the latter being preferred.

In order to be able to also solubilize the quinone, the polarity of the solvent mixture is preferably not too high. Hence, there is preferably at least 30 wt % of non-polar solvent in the organic solvents mixture, and more preferably at least 40 wt %. Generally, there is not more than 80 wt % of this non-polar solvent, preferably not more than 60 wt % of it in the organic solvents mixture.

The non-polar solvent preferably is an aromatic solvent or a mixture of aromatic solvents. Aromatic solvents are for instance selected from benzene, toluene, xylene, tert-butylbenzene, trimethylbenzene, tetramethylbenzene, naphthalene, methylnaphthalene mixtures of polyalkylated benzenes, and mixtures thereof. The commercially available aromatic hydrocarbon solvent of type 150 from the Solvesso® series (or equivalent from other supplier) gives good results. S-150 (Solvesso®-150; CAS no. 64742-94-5) is known as an aromatic solvent of high aromatics which offer high solvency and controlled evaporation characteristics that make them excellent for use in many industrial applications and in particular as process fluids. The Solvesso® aromatic hydro-carbons are available in three boiling ranges with varying volatility, e.g. with a distillation range of 165-181° C., of 182-207° C. or 232-295° C. They may be obtained also naphthalene reduced or as ultra-low naphthalene grades. Solvesso® 150 (S-150) is characterized as follows: distillation range of 182-207° C.; flash point of 64° C.; aromatic content of greater than 99% by wt; aniline point of 15° C.; density of 0.900 at 15° C.; and an evaporation rate (nButAc=100) of 5.3.

As explained above, the hydrogenation reaction takes place in the presence of a catalyst (like for instance the one object of WO 2015/049327 in the name of the Applicant) and as for instance described in WO 2010/139728 also in the name of the applicant (the content of both references being incorporated by reference in the present application). Typically, the hydrogenation is conducted at a temperature of at least 45° C. and preferably up to 120° C., more preferably up to 95° C. or even up to 80° C. only. Also typically, the hydrogenation is conducted at a pressure of from 0.2 to 5 bars. Hydrogen is typically fed into the vessel at a rate of from 650 to 750 normal m3 per ton of hydrogen peroxide to be produced.

The oxidation step may take place in a conventional manner as known for the AO-process. Typical oxidation reactors known for the anthraquinone cyclic process can be used for the oxidation. Bubble reactors, through which the oxygen-containing gas and the working solution are passed co-currently or counter-currently, are frequently used. The bubble reactors can be free from internal devices or preferably contain internal devices in the form of packing or sieve plates. Oxidation can be performed at a temperature in the range from 30 to 70° C., particularly at 40 to 60° C. Oxidation is normally performed with an excess of oxygen, so that preferably over 90%, particularly over 95%, of the alkyl anthrahydroquinones contained in the working solution in hydroquinone form are converted to the quinone form.

After the oxidation, during the purification step, the hydrogen peroxide formed is separated from the working solution generally by means of an extraction step, for example using water, the hydrogen peroxide being recovered in the form of a crude aqueous hydrogen peroxide solution. The working solution leaving the extraction step is then recycled into the hydrogenation step in order to recommence the hydrogen peroxide production cycle, eventually after having been treated/regenerated.

In a preferred embodiment, after its extraction, the crude aqueous hydrogen peroxide solution is washed several times i.e. at least two times consecutively or even more times as required to reduce the content of impurities at a desired level.

The term “washing” is intended to denote any treatment, which is well known in the chemical industry (as disclosed in GB841323A, 1956 (Laporte), for instance), of a crude aqueous hydrogen peroxide solution with an organic solvent which is intended to reduce the content of impurities in the aqueous hydrogen peroxide solution. This washing can consist, for example, in extracting impurities in the crude aqueous hydrogen peroxide solution by means of an organic solvent in apparatuses such as centrifugal extractors or liquid/liquid extraction columns, for example, operating counter-current wise. Liquid/liquid extraction columns are preferred. Among the liquid/liquid extraction columns, columns with random or structured packing (like Pall rings for instance) or perforated plates are preferred. The former are especially preferred.

In a preferred embodiment, a chelating agent can be added to the washing solvent in order to reduce the content of given metals. For instance, an organophosphorus chelating agent can be added to the organic solvent as described in the above captioned patent application EP 3052439 in the name of the Applicant, the content of which is incorporated by reference in the present application.

The expression “crude aqueous hydrogen peroxide solution” is intended to denote the solutions obtained directly from a hydrogen peroxide synthesis step or from a hydrogen peroxide extraction step or from a storage unit. The crude aqueous hydrogen peroxide solution can have undergone one or more treatments to separate out impurities prior to the washing operation according to the process of the invention. It typically has an H₂O₂ concentration within the range of 30-50% by weight.

The solvents of the invention make it is possible to achieve a higher solubility and thus there is less polar solvent needed to achieve a higher partition coefficient. With this higher partition coefficient it is possible to reduce the capex (capital expenditure) required for the extraction sector.

The solvents of the invention are particularly suitable for the manufacture of hydrogen peroxide by the AO-process wherein said process has a production capacity of hydrogen peroxide of up to 100 kilo tons per year (ktpa). Preferably said process is a small to medium scale AO-process operated with a production capacity of hydrogen peroxide of up to 50 kilo tons per year (ktpa), and more preferably with a production capacity of hydrogen peroxide of up to 35 kilo tons per year (ktpa), and in particular a production capacity of hydrogen peroxide of up to 20 kilo tons per year (ktpa). The dimension ktpa (kilo tons per annum) relates to metric tons.

A particular advantage of such a small to medium scale AO-process is that the hydrogen peroxide can be manufactured in a plant that may be located at any, even remote, industrial end user site and the solvents of the invention are therefore especially suitable. It is namely so that since their partition coefficient is more favourable, less emulsion is observed in the process and a purer H₂O₂ solution can be obtained (namely containing less TOC) and this for a longer period of time compared to when solvents known from prior art are used. In a preferred sub-embodiment of the invention, the working solution is regenerated either continuously or intermittently, based on the results of a quality control, regeneration meaning conversion of certain degradates, like epoxy or anthrone derivatives, back into useful quinones. Here also, the solvents of the invention are favourable because the quality of the H₂O₂ solution can be maintained within the specifications namely in terms of TOC for a longer period of time.

The following examples illustrate some preferred embodiments of the present invention.

### EXAMPLE 1: SYNTHESIS OF 2,2,6-TRIMETHYLCYCLOHEXANECARBONITRILE (C10A)

Step 1

To a 6 L double-jacketed reactor equipped with mechanical stirring, a condenser linked to nitrogen arrival, temperature probe and additional funnel were added toluene (3 L, 3 vol), geranic acid (1 Kg, 1030 mL, 5.05 mol, 1 eq.) and more toluene (1 L, 1 vol). The yellow solution was heated to 110° C. then H3PO4 (85% purity, 103.7 mL, 174.7 g, 1.52 mol, 0.3 eq) was added. The orange brown solution was then stirred at 110° C. for 6 h (NMR monitoring). The reaction mixture was cooled down to 20° C., and neutralized with brine solution (2 L containing 376 g of NaCl). After neutralization and decantation, the organic phase was washed with water (1 L), concentrated under reduced pressure to afford the final product as a white solid. In some cases, if there was too much starting material left, a slurry in hexane (2 vol) at rt (room temperature) followed by filtration gave the pure desired product as a white solid

832 g of a white solid corresponding to the cyclohexenecarboxylic acid intermediate with a NMR purity (α, α, α-trifluorotoluene as internal standard) of 74% were obtained.

Step 2

In a 1 L double jacketed stainless steel hydrogenator equipped with H₂ and N2 entry, a mechanical stirring and a condenser were added cyclohexene carboxylic acid intermediate (230 g, 1.35 mol, 1 eq.), PtO2 (3.11 g, 0.014 mol, 0.01 eq.) and acetic acid (604 g, 575 mL, 2.5 vol.). The reaction mixture was stirred at rt, and Continuous H2 flow (1 bar) was sent to the reactor for 1h. The mixture was then heated to 50° C. for one more hour. The reaction mixture was then cooled to 20° C. and concentrated under vacuum to afford the desired product as colorless oil which crystallized to white solid with time.

240 g of colorless oil which crystallized to white solid with time, corresponding to cyclohexane carboxylic acid intermediate with a NMR purity (trifluorotoluene as standard) of 88.8% were obtained.

Step 3

In a 3 L double jacketed reactor under nitrogen equipped with a mechanical stirring, an introduction pump and a condenser connected to a scrubber filled with NaOH 15%, cyclohexane carboxylic acid intermediate (500.60 g, 2.94 mol, 1 eq.) was added. The product was heated to 50° C. then SOCl2 (371 g, 227 mL, 3.08 mol, 1.05 eq.) was added dropwise by the pump over one hour, while HCl is degazed and trapped by the scrubber. The reaction mixture was stirred at 80° C. for one hour, and then heated to 130° C. while sulfolan (568 g, 450 mL, 0.9 vol.) was added to the mixture. In parallel, in another 1 L double jacketed reactor, a solution of sulfamide (342 g, 3.52 mol, 1.2 eq.) in sulfolane (1388 g, 1100 mL, 2.2 vol.) was prepared and heated at 50° C. This solution is then added dropwise via an addition pump to the reaction mixture at 130° C. over one hour. The mixture was stirred at 130° C. for 2h then cooled to 20° C. and quenched with NaOH 20% solution (764 g, 3.82 mol, 1.3 eq.). In a 6 L reactor the mixture was diluted with water (750 g) and extracted three times with a mixture hexane/MTBE 3/2 (3*1000 mL). The combined organic phases were washed with water three times (3*1400 g) and concentrated under vacuum to afford a dark orange liquid (388 g, 83.9% yield). The crudes of three cyanation batches were purified together by vacuum distillation (90° C., 10 mbar) to afford the pure desired product.

830 g of a colorless liquid corresponding to the C10A solvent (2,2,6-trimethylcyclohexanecarbonitrile (C10A)) with a GC purity (area) superior to 99% with its isomers were obtained.

### EXAMPLE 2: SYNTHESIS OF 2,2,5,6-TETRAMETHYLCYCLOHEXANE-1-CARBONITRILE (C11A)

Step 1

In a 10 L reactor, 2-methylacetoacetate (3.2 mol: 455 g) is diluted in absolute ethanol (3.5 L) before adding sodium ethanolate (1.05 equivalents: 224 g) over a period of 30 minutes under an inert atmosphere. The reaction medium is then stirred mechanically for 1 h at 25° C. before being cooled to −10° C. A solution of 1-chloro-3-methyl-2-butene (1.05 equivalents: 104.5 g) diluted in 1 L of absolute ethanol is added. The reaction medium is brought to ambient temperature and is stirred overnight. The reaction medium is filtered through celite and concentrated under reduced pressure, yielding ethyl 2-acetyl-2,5-dimethylhex-4-enoate, isolated in the form of a yellow oil (yield: quantitative).

Step 2

In a 10 L reactor, 47% aqueous potassium hydroxide solution (2 L) is diluted with water (2 L) and ethanol (2 L). Ethyl 2-acetyl-2,5-dimethylhex-4-enoate (2.8 mol: 600 g) is added, and the reaction mixture is refluxed for 6 h. After cooling, the reaction medium is diluted with cyclohexane (2 L) and an 18% sodium chloride solution (2 L) is added. The aqueous phase is extracted with cyclohexane (2 L), and the combined organic phases are successively washed with a solution of 18% NaCl (2 L), a solution of 3.5% HCl (500 mL) to reach a pH of 7 dried over magnesium sulphate, filtered and concentrated under reduced pressure. The crude reaction product is finally purified by distillation under reduced pressure (15 bar, 65° C.), resulting in isolated 3,6-dimethylhept-5-en-2-one in the form of a colorless oil (yield over 2 steps: 60%).

Step 3

In a 10 L reactor, triethylphosphonoacetate (1.1 equivalents: 423 g) is diluted in THF (3.5 L). The solution is cooled to −10° C. before adding 60% NaH diluted in oil (1.2 equivalents: 83 g) over a period of 30 minutes. A solution of 3,6-dimethylhept-5-en-2-one 1 (1.7 mol: 240 g) diluted in THF (300 mL) is added and the reaction medium is brought to room temperature and mechanically stirred overnight. A solution of 18% NaCl (2 L) and cyclohexane (2 L) is added to the reaction medium. The aqueous phase is extracted with cyclohexane (1 L), and the combined organic phases are successively washed with 18% NaCl solution, dried over magnesium sulfate, filtered and concentrated under reduced pressure. The crude reaction medium is finally purified by flash chromatography on silica gel (eluent: cyclohexane/MTBE 100%→93-7%) to produce, after evaporation under reduced pressure, (Z/E) ethyl 3,4,7-trimethylocta-2,6-dienoate which is isolated as a yellow oil (yield: 84%); the product has been characterized by proton NMR

Step 4

In a 10 L reactor, boron trifluoride etherate (1.27 equivalents: 416 g) is diluted in toluene (3 L). A solution of ethyl (Z/E) ethyl 3,4,7-trimethylocta-2,6-dienoate (2.3 mol: 486 g) diluted in toluene (1 L) is added. The reaction medium is heated at 50° C. for 2 h before being quenched with iced water in another 101 reactor. Toluene (500 ml) used to clean the first reactor is added to the reaction medium. A solution of 18% NaCl (500 mL) and MTBE (500 mL) is added to make the reaction medium less turbid, as well as toluene (500 mL). The aqueous phase is separated, and the organic phase is washed with 18% NaCl solution (1.5 L). The combined aqueous phases are extracted with MTBE (500 mL), and the combined organic phases are successively washed with 26% NaCl solution (3 L), dried over magnesium sulfate, filtered and concentrated under reduced pressure. The crude reaction product is finally purified by distillation under reduced pressure (3 mbar, 75-77° C.), yielding ethyl 2,3,6,6-tetramethylcyclohex-2-ene-1-carboxylate isolated in the form of a colorless oil (yield: 91%); the product has been characterized by proton NMR and mass spectrometry.

Step 5

In a 1 L autoclave, platinum dioxide (0.04 equivalents: 5 g) is added to ethyl 2,3,6,6-tetramethylcyclohex-2-ene-1-carboxylate (0.52 mol: 110 g) diluted in acetic acid (500 mL). The reaction medium is put under a constant pressure of 11 bar H2 for 8 h, filtered and concentrated under reduced pressure. The solution is then diluted in MTBE (150 mL) and successively washed with water (50 mL), a 26% NaCl solution (2×50 mL), dried over magnesium sulfate and concentrated under reduced pressure to yield ethyl 2,5,6-tetramethylcyclohexane-1-carb oxylate isolated as a colorless oil (yield: 93%); the product has been characterized by proton NMR.

Step 6

In a 1 L autoclave, potassium hydroxide (5.2 equivalents: 185.5 g) is added to a solution of ethyl 2,2,5,6-tetramethylcyclohexane-1-carboxylate (0.64 mol: 135 g) diluted in methanol (400 mL). The reaction medium is heated to 175° C. (˜14 bar) and stirred for 6h30. The reaction medium is concentrated under reduced pressure before dissolving the sodium hydroxide in water (75 ml). Iced water is added to the medium and acidified by adding 36% HCl. The aqueous phase is extracted with ethyl acetate (2×250 mL), and the combined organic phases are successively washed with 26% NaCl solution (100 mL), dried over magnesium sulphate and concentrated under reduced pressure, resulting in 2,2,5,6-tetramethylcyclohexane-1-carboxylic acid isolated as a yellow oil (yield: 97%); the product has been characterized by proton IR spectroscopy and mass spectrometry

Step 7

In a 1 L flask, thionyl chloride (1.24 equivalents: 187.6 g) is added to 2,2,5,6-tetramethylcyclohexanecarboxylic acid (1.27 mol: 234 g).). The reaction medium, equipped with a scrubber, is stirred for one hour before being refluxed for another hour. After cooling, the reaction medium is concentrated under reduced pressure to evaporate the excess thionyl chloride. The crude reaction medium is finally purified by distillation under reduced pressure (145 mbar, 163° C.) resulting in 2,2,5,6-tetramethylcyclohexanecarbamoyl chloride isolated in the form of a colorless oil (yield: 90%); the product has been characterized by proton NMR and mass spectrometry

Step 8

In a 1 L flask, the sulfonamide (1.2 equivalents: 85.5 g) is added to the 2,2,5,6-tetramethylcyclohexane-1-carbonyl chloride (0.74 mol: 150 g) diluted in the sulfolane (400 mL) under an inert atmosphere. The reaction medium is heated at 180° C. with mechanical stirring for 3 h before being quenched with 0.7 M sodium hydroxide (3.5 L) and brine (26% sodium chloride solution) (300 ml) is added. The aqueous phase is extracted with hexane-MTBE (3:2) (5×500 ml), and the combined organic phases are successively washed with water (4×1 L), dried over magnesium sulphate and concentrated under reduced pressure. The crude reaction medium is finally purified by distillation under reduced pressure (7 mbar, 86-87° C.), resulting in the isolated 2,2,5,6-tetramethylcyclohexane-1-carbonitrile C11A in the form of a colorless oil (yield: 87.degree. %). the product has been characterized by proton NMR and mass spectrometry

### EXAMPLE 3: SYNTHESIS OF 2,2,5,6-TETRAMETHYLCYCLOHEXANECARBONITRILE (C11A)

Steps 1 to 6 are identical to those of Example 2 but steps 7 and 8 have been regrouped in a single one pot synthesis as follows:

In a 1 L flask, thionyl chloride (1.24 equivalents: 187.6 g) is added to 2,2,5,6-tetramethylcyclohexane-1-carboxylic acid (1.27 mol: 234 g).). The reaction medium, equipped with a scrubber, is stirred for one hour before being heated.

Then dilution with sulfolane (same concentration as in Example 2), introduction of sulfamide (same amount as in Example 2) and performing all reaction steps exactly as in Example 2. After concentration of the reaction medium, a liquid with a mass balance of 84% molar is obtained and its structure was confirmed by NMR analysis.

### EXAMPLE 4: SYNTHESIS OF 1,2,2,4-TETRAMETHYLCYCLOHEXANECARBONITRILE (C11B)

Step 1: Diels-Alder Reaction

In a three-necked flask equipped with a mechanical stirrer and a condenser, 50 g of BoB(Ac)4 (182 mmol) and 147 g of methacrylic acid (1.7 mol) were introduced.

After dissolution, the medium was diluted with 600 ml of dry THF and 150 g of 2,4-dimethyl-1,3-pentadiene (1.56 mol) were added. The whole was heated at 60° C. during 16h and then, the solvent was evaporated under vacuum.

500 ml of water were added to hydrolyze the catalyst (BoB(Ac)4), and then the aqueous phase was extracted with ethyl acetate (4×200 ml).

The organic phase was washed with 20 ml of a NaCl solution (26%) and then dried over magnesium sulfate and concentrated under vacuum.

240 g of solid were obtained.

This solid was dissolved/suspended in 1 L of petroleum ether and brought to reflux.

Then it was allowed to cool to room temperature, and placed at 5° C. overnight to finish crystallization.

It was then filtered to obtain 170 g of 1,2,2,4-tetramethylcyclohex-3-enecarboxylic acid and its 1,3,3,5 isomer (in ratio 3/1) as proved by NMR analysis.

Step 2: Hydrogenation

In a 2 L jacketed reactor, 165 g of the 1,2,2,4-tetramethylcyclohex-3-enecarboxylic acid (0.9 moles) were introduced and then diluted with 800 ml of glacial acetic acid.

10 g of platinum oxide were added and nitrogen was circulated to purge the atmosphere.

A slight overpressure (100 mbar) of H2 was then applied while stirring at 1500 RMP and monitoring the H2 consumption with a mass flow meter.

After 1.5h hydrogen consumption was no longer observed and thus the reactor was purged with nitrogen for 15 min.

The reaction medium was then filtered, the solvent evaporated, and the filter residue dissolved in 750 ml of ethyl acetate, the organic phase washed with 250 ml of water and 2×250 ml of 26% NaCl.

After drying, 158 g of oil was obtained, which was found to be 1,2,2,4-tetramethylcyclohexyl carboxylic acid and its 1,3,3,5 isomer (in ratio 3/1) by NMR analysis (no purification necessary).

Step 3: Chlorination

In a three-necked 1 L flask equipped with a thermocouple, a condenser and under an inert atmosphere, 150 g of 1,2,2,4-tetramethylcyclohexanecarboxylic acid were introduced and 1.2 equivalents (1.03 mol) of thionyl chloride were added. (143 g).

At the outlet of the condenser, a guard vessel under nitrogen followed by a trap (15% NaOH) was placed with stirring to trap the HCl released.

After a few minutes, an endotherm was observed (medium at 5° C.) with significant degassing.

The medium was then heated at about 75° C. (reflux of thionyl chloride) until slight reflux for one hour.

The product obtained was first distilled at atmospheric pressure to remove the remaining thionyl chloride, then the chloride(s) were isolated at 141° C./60 mbar. 151 g of slightly yellow liquid was obtained (yield 86% in 1,2,2,4- and 1,3,3,5-tetramethylcyclohexanecarbonyl chloride).

Step 4: Cyanation

In a two-necked 2 L flask with mechanical stirring and under an inert atmosphere, 125 g of sulfonamide (1.3 moles) were introduced.

Then, it was diluted with 1 L of sulfolane previously melted at 40° C.

200 g of 1,2,2,4- and 1,3,3,5-tetramethylcyclohexanecarbonyl chloride (˜1 mole) were added while stirring for 30 min.

It was then heated at 140° C. for 4h (conversion followed by GC).

The medium was cooled and then poured into a solution of NaOH 100 g/5 L of water.

The organic phase was extracted in 4×1 of a cyclohexane/MTBE mixture (2/1).

The organic phases were combined, washed with 3×500 ml of water and 500 ml of 26% NaCl.

The crude reaction medium was dried over sulfate magnesium before being concentrated to give an oil (170 g) before distillation.

Distillation at 77-79° C./5 mBar gave 160 g of 1,2,2,4-tetramethylcyclohexanecarbonitrile (C11B) and its 1,3,3,5 isomer.

### EXAMPLE 5: SOLUBILITY TESTS OF HYDROGENATED QUINONES IN DIFFERENT SOLVENT MIXTURES

The determination of the QH solubility was performed on synthetic EQ/ETQ working solutions. These quinones mixed in the tested solvents have been hydrogenated to a fixed level and cooled down successively to 3 different temperatures before the measurement (min. 3 hours to stabilize the system between each measurement). The conditions applied for these tests were:

| EQ concentration | 100 | g/kg |
|---|---|---|
| ETQ concentration | 140 | g/kg |
| Polar solvent | variable (*) | |
| Level of hydrogenation | 10.8 Nl H2/kg WS (~116 g of QH/kg | |
| | of WS or a TL (Test Level) of 16.3 | |
| | g of H2O2/kg of WS (=maximum | |
| | theoretical value of TL if all QH dissolved)) | |
| Temperature of | 75° | C. |
| hydrogenation | | |

| | |
|---|---|
| Temperatures of | indicated in Table 1 as temperature |
| precipitation | at which QH is measured |

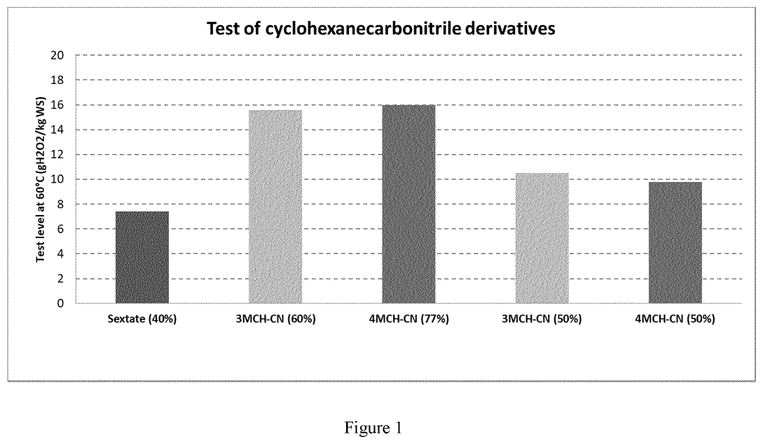

(*) the polar solvents tested were sextate, decanenitrile, 2,2,6-trimethylcyclohexanecarbonitrile (C10A), 2,2,5,6-tetramethylcyclohexanecarbonitrile (C11A) and 1,2,2,4-tetramethylcyclohexylcarbonitrile (C11B). They were used in mixture with S-150 in the ratios indicated in Table 1, Table 1-2, Table 2 and FIG. 1 attached, which also shows the results obtained. In this Figure, Kb stands for the weight partition coefficient of hydrogen peroxide between the water and the working solution (mixture of quinones and organic solvents). It is calculated using the following formula:

Kb=(g H2O2/kg aqueous phase)/(g H2O2/kg organic phase)

The Tables and FIG. 1 demonstrate the very high potential of cyclohexanecarbonitrile structures versus linear nitriles like decanenitrile, and especially of solvents C11A and C11B which when used in a ratio of 77% and 70% respectively, even lead to complete solubility of the QH at 60° C. (and hence, to a TL of 16.3 as calculated above).

The maximum solubility of a hydrogenated quinone (QH) in a solvent mixture is directly correlated with the productivity of the working solution. The higher is the QH solubility, the higher will be the theoretical quantity of hydrogen peroxide achievable per kg of WS (Productivity). These theoretical values, designated by the terms “Test level (gH2O2/kg of WS) measured at . . . ” in Table 1, were calculated as follows:

1 mole (240 g) ETQH (which actually is the QH in our Examples) per kg of WS will produce 1 mole (34 g) of H₂O₂ per kg of WS. Hence, the test level in our Examples equals: 34*QH/240.

Again, the values obtained with 2,2,6-trimethylcyclohexanecarbonitrile (C10A) are much higher (almost the double in fact) than with sextate or a linear nitrile like decanenitrile, and with solvent C11A, they even reach the absolute maximum theoretical value.

Table 2 attached also shows that solvents C11A and C11B are less soluble in H₂O₂ than solvent C10A (as indicated by the lower level of TOC in the H₂O₂ obtained), and hence allow reaching a higher purity level of the H2O2.

TABLE 1

| | | | | 2,2,6-trimethyl- | 2,2,6-trimethyl- |
|---|---|---|---|---|---|
| | | Methylcyclohexyl | | cyclohexane- | cyclohexane- |
| | | acetate | Decanenitrile | carbonitrile | carbonitrile |
| | Solvant | Sextate (40%) | Decanenitrile (65%) | 3MCH-CN (60%) | 3MCH-CN (50%) |
| Mass ratio of polar solvent in solvent | % | 40 | 35 | 60 | 50 |
| Mass ratio of S150 in solvent | % | 60 | 65 | 40 | 50 |
| Kb of the solvents mix | | 176 | 171 | 171 | 247 |
| Approx density of polar solvents at amb T° (kg/l) | | 0.94 | 0.83 | 0.89 | 0.89 |
| QH (g/kg) measured at . . . | 50° C. | | | | |
| | 55° C. | | | | |
| | 60° C. | 52 | 62.2 | 110 | 73.9 |
| | 65° C. | 60.8 | 71.5 | full soluble | 80.3 |
| | 70° C. | 68 | 78.6 | full soluble | full soluble |
| Test level (gH2O2/kg) measured at . . . | 50° C. | | | | |
| | 55° C. | | | | |
| | 60° C. | 7.4 | 8.8 | 15.6 | 10.5 |
| | 65° C. | 8.6 | 10.1 | full soluble | 11.4 |
| | 70° C. | 9.6 | 11.1 | full soluble | full soluble |

| | | 2,3,6,6-tetramethyl- | 2,3,6,6-tetramethyl- |
|---|---|---|---|
| | | cyclohexane- | cyclohexane- |
| | | carbonitrile | carbonitrile |
| | Solvant | 4MCH-CN (77%) | 4MCH-CN (50%) |
| Mass ratio of polar solvent in solvent | % | 77 | 50 |
| Mass ratio of S150 in solvent | % | 23 | 50 |
| Kb of the solvents mix | | 176 | 300 |
| Approx density of polar solvents at amb T° (kg/l) | | 0.895 | 0.895 |
| QH (g/kg) measured at . . . | 50° C. | 84.2 | 54.4 |
| | 55° C. | 90.5 | 60.4 |
| | 60° C. | full soluble | 69.1 |
| | 65° C. | full soluble | 75.9 |
| | 70° C. | full soluble | 104.5 |
| Test level (gH2O2/kg) measured at . . . | 50° C. | 11.9 | 7.7 |
| | 55° C. | 12.8 | 8.6 |
| | 60° C. | full soluble | 9.8 |
| | 65° C. | full soluble | 10.8 |
| | 70° C. | full soluble | 14.8 |

TABLE 1-2

| | | 1,2,2,4 | 1,2,2,4 |
|---|---|---|---|
| | | tetramethylcyclo | tetramethylcyclo |
| | | hexancarbonitrile | hexancarbonitrile |
| | Solvant | (C11B) | (C11B) |
| Mass ratio of polar | % | 70 | 52 |
| solvent | | | |
| Mass ratio of S150 | % | 30 | 48 |
| Partition coefficient of | | 176 | 300 |
| solvent Kb | | | |
| QH (g/kg) measured | 55° C. | 109.4 | 66.6 |
| at . . . | 60° C. | full soluble | 76.4 |
| | 65° C. | full soluble | 106.6 |
| | 70° C. | full soluble | full soluble |
| Test level (gH2O2/kg) | 55° C. | 15.5 | 9.4 |
| measured at . . . | 60° C. | full soluble | 10.8 |
| | 65° C. | full soluble | 15.1 |
| | 70° C. | full soluble | full soluble |

TABLE 2

| | | 2,2,6-trimethylcyclohexane- | | | | 2,2,5,6-tetramethylcyclohexane- | | | | 1,2,2,4-tetramethylcyclohexane- | | | |
|---|---|---|---|---|---|---|---|---|---|---|---|---|---|
| | | carbonitrile (C10A) | | | | carbonitrile (C11A) | | | | carbonitrile (C11B) | | | |
| | | C10H17N | | | | C11H19N | | | | C11H19N | | | |
| Polar | Solvesso | H2O2 in | | | TOC in | H2O2 in | | | TOC in | H2O2 in | | | TOC in |
| solvent | 150 | OP | | | H2O2 | OP | | | H2O2 | OP | | | H2O2 |
| (%) | (%) | (g/kg) | Kb | Kc | (ppm) | (g/kg) | Kb | Kc | (ppm) | (g/kg) | Kb | Kc | (ppm) |
| 30 | 70 | 0.9821 | 538 | | 928 | 0.919 | 575 | | 281 | | | | |
| 40 | 60 | 1.466 | 360 | | 1042 | 1.271 | 416 | | 268 | 1.632 | 333 | | 308 |
| 50 | 50 | 2.135 | 247 | | 1248 | 0 | 0 | | 269 | 2.487 | 217 | | 328 |
| 60 | 40 | 3.08 | 171 | | 1456 | 2.332 | 238 | | 274 | 3.167 | 171 | | 280 |
| 70 | 30 | 3.871 | 136 | | 1684 | 2.786 | 199 | | 291 | 3.805 | 142 | | 290 |

1-15. (canceled)

16. A process for manufacturing a substituted cyclohexanecarbonitrile said process comprising the following steps:
reacting the corresponding substituted cyclohexanecarboxylic acid with thionyl chloride to make the corresponding acyl chloride; and simultaneously or subsequently
reacting the chloride with sulfonamide in sulfolane as solvent to make the substituted cyclohexanecarbonitrile.

17. The process according to claim 16, wherein the substituted cyclohexanecarboxylic acid is obtained by hydrogenating one of the corresponding substituted cyclohexenecarboxylic acids with hydrogen gas in the presence of a hydrogenation catalyst.

18. The process according to claim 17, wherein the catalyst is PtO2 and wherein the hydrogenation takes place in glacial acetic acid as solvent.

19. The process according to claim 17, wherein the substituted cyclohexenecarboxylic acid is obtained by cyclization of the corresponding linear acid in the presence of a catalyst.

20. The process according to claim 19, wherein the linear acid is geranic acid, the catalyst is phosphoric acid in toluene and the resulting substituted cyclohexanecarbonitrile is 2,2,6-trimethylcyclohexanecarbonitrile (C10A).

21. The process according to claim 17, wherein the substituted cyclohexenecarboxylic acid is obtained by a Diels-Alder reaction between a conjugated diene and an unsaturated carboxylic acid in the presence of a Lewis acid catalyst.

22. The process according to claim 21, wherein:
the conjugated diene is 2,4-dimethylpenta-1,3-diene, the unsaturated carboxylic acid is methacrylic acid, the Lewis acid catalyst is BoB(Ac)4, THF is used as solvent and the resulting substituted cyclohexanecarbonitrile is 1,2,2,4-tetramethylcyclohexylcarbonitrile (C11B) eventually comprising its 1,3,3,5 isomer; or
the conjugated diene is 2,4-dimethylpenta-1,3-diene, the unsaturated carboxylic acid is crotonic acid, the Lewis acid catalyst is BoB(Ac)4, THF is used as solvent and the desired resulting substituted cyclohexanecarbonitrile is 2,2,4,6-tetramethylcyclohexylcarbonitrile (C11C) eventually comprising its 2,3,3,5 isomer; or
the conjugated diene is 2,3-dimethylbuta-1,3-diene, the unsaturated carboxylic acid is tiglic acid, the Lewis acid catalyst is BoB(Ac)4, THF is used as solvent and the resulting substituted cyclohexanecarbonitrile is one of the stereoisomers of 1,2,4,5-tetramethylcyclohexanecarbonitrile (C11D); or
the conjugated diene is 2,3-dimethylbuta-1,3-diene, the unsaturated carboxylic acid is angelic acid, the Lewis acid catalyst is BoB(Ac)4, THF is used as solvent and the resulting substituted cyclohexanecarbonitrile is another stereoisomer of 1,2,4,5-tetramethylcyclohexenecarbonitrile (C11E); or
the conjugated diene is 2,4-dimethylpenta-1,3-diene, the unsaturated carboxylic acid is tiglic acid, the Lewis acid catalyst is BoB(Ac)4, THF is used as solvent and the resulting substituted cyclohexenecarbonitrile is 1,2,3,3,5-pentamethylcyclohexanecarbonitrile (C12A).

23. The process according to claim 16, wherein the substituted cyclohexanecarboxylic acid is obtained by hydrolysing the corresponding substituted cyclohexane ester, which substituted cyclohexane ester is obtained by hydrogenating the corresponding substituted cyclohexene ester with hydrogen gas in the presence of a hydrogenation catalyst.

24. The process according to claim 23, wherein the substituted cyclohexane ester is ethyl 2,2,5,6-tetramethylcyclohexanecarboxylate, the substituted cyclohexene ester is ethyl 2,3,6,6-tetramethylcyclohex-2-enecarboxylate and the resulting substituted cyclohexanecarbonitrile is 2,2,5,6-tetramethylcyclohexanecarbonitrile (C11A).

25. The process according to claim 24, wherein the ethyl 2,3,6,6-tetramethylcyclohex-2-enecarboxylate is been obtained through the following reaction steps:
a second order nucleophilic substitution reaction (SN2) on ethyl 3-oxo-2-methylbutanoate (so called 2-methylacetoacetate) with 1-chloro-3-methyl-2-butene in alkaline medium and the subsequent decarboxylation of the alpha-ketoacid to afford the compound 3,6-dimethylhept-5-en-2-one;
a Horner-Wadsworth-Emmons (HWE) reaction on the obtained 3,6-dimethylhept-5-en-2-one with triethylphosphonoacetate and sodium hydride to form the corresponding ester ethyl 3,4,7-trimethylocta-2,6-dienoate;
the cyclization of ethyl 3,4,7-trimethylocta-2,6-dienoate with a Lewis acid catalyst or with phosphoric acid in toluene to obtain ethyl 2,3,6,6-tetramethylcyclohex-2-enecarboxylate.

26. A substituted cyclohexanecarbonitriles obtainable by a process according to claim 16.

27. A substituted cyclohexanecarbonitrile having the formula C11A, C11B, C11C, C11D, C11E or C12A.

28. A process for manufacturing an aqueous hydrogen peroxide solution comprising the following steps:
hydrogenating a working solution which comprises an alkylanthraquinone and/or tetrahydroalkylanthraquinone and a mixture of a non-polar organic solvent and a polar organic solvent;
oxidizing the hydrogenated working solution to produce hydrogen peroxide; and
isolating the hydrogen peroxide,
wherein the polar organic solvent is the substituted cyclohexanecarbonitrile according to claim 26.

29. The process according to 28, said process having a production capacity of hydrogen peroxide of up to 100 kilo tons per year.

30. The process according to claim 28, said process being operated in a plant located at an industrial end user site.
